(19) United States
(12) Patent Application Publication
(10) Pub. No.: US 2008/0119690 A1
(43) Pub. Date: May 22, 2008

(54) PENILE ERECTION ASSISTING DEVICE

(76) Inventor: Jean Comeau, Quebec (CA)

Correspondence Address: BROUILLETTE & PARTNERS, METCALFE TOWER, 1550 METCALFE STREET, SUITE 800, MONTREAL, QC, H3A-1X6, omitted

(21) Appl. No.: 11/673,780

(22) Filed: Feb. 12, 2007

(30) Foreign Application Priority Data
Nov. 22, 2006 (CA) 2,568,649

(51) Int. Cl. A61B 18/18

(52) U.S. Cl. 600/39

(57) ABSTRACT

A device for assisting in and maintaining an erection of the human penis is provided. The device generally comprises a flexible elongated element and an elastic elongated element mounted to and extending from each end of the flexible elongated element in order to define a loop. The device is adapted to be worn during sexual intercourse in order to assist in and generally maintain the erection of a human penis.

### CROSS-REFERENCE TO RELATED APPLICATIONS

There are no cross-related applications.

### FIELD OF THE INVENTION

The present invention generally pertains to devices for assisting in and maintaining erection of the human penis.

### BACKGROUND OF THE INVENTION

For various physical and psychological reasons, some males are not able to attain and/or maintain penile erection, rendering satisfactory sexual intercourse difficult or worse, impossible. Even though most males may attain and maintain penile erections which are adequate for sexual intercourse, the experience thereof could be enhanced for the male and his female partner so that the act of intercourse would be more satisfactory to both of them.

To aid those incapable of normal penile erection and to enhance the sexual experience of others, a number of devices have been invented to assist, maintain or substitute for penile erection. In some cases, surgical implanting of prosthetic devices have been utilized. Surgical procedures are generally avoided by most males. Such surgical procedures are usually expensive and are not without some degree of risk.

Other solutions to the problem of inadequate penile erection are provided by apparatus which may be worn and utilized by the male without surgical procedure. Some of these provide artificial support or rigidity by providing members to surround and/or support the penis and which are normally attached to the body with a harness-like arrangement. Examples of such devices may be seen in U.S. Pat. Nos. 4,022,196; 4,381,000; 4,429,689 and 4,488,541. These devices are rather cumbersome and relatively uneasy to use.

Other devices, proposed to assist in or maintain penile erection, function by exerting pressure to or around the penis in order to control the return flow of blood therefrom, thus maintaining the penis in an erect condition. Some of these devices, such as those presented in U.S. Pat. Nos. 3,845,760; 3,461,863; and 3,612,047, function essentially as a tourniquet. Thus, there are some dangers involved if blood circulation is too severely restricted. In addition, the tourniquet-like device, while restricting blood flow, may also restrict and/or impede ejaculation, causing physical harm and even pain.

Because of some of the problems inherent with tourniquet-like devices, other devices have been proposed which restrict blood flow from the penis but do not totally surround or clamp the penis so as to avoid the inherent problems thereof. Examples of these devices may be seen in U.S. Pat. Nos. 3,511,230; 3,633,572 and 4,440,183. While these devices are inherently safer, they are more difficult to maintain in proper position and the means for maintaining them in proper position is sometimes uncomfortable and detract from the user's sexual urge and enjoyment.

In order to solve the problems caused by the devices presented above, some more recent devices have been proposed wherein pressure is only applied to the top portion of the base of the penis. Examples of such devices may be seen in U.S. Pat. Nos. 4,440,183 and 4,643,175. By doing so, blood is generally allowed to flow in the penis but is partially restricted from flowing out. Hence, these devices assist in and generally maintain the erection. Yet, in these devices, the member which is located on the top portion of the base of the penis is generally rigid in nature. Moreover, the rigid member generally has a tendency to move during sexual intercourse and therefore can cause injuries to the penis and to the testicles.

Obviously, with the continued development and proliferation of devices to aid in attaining and maintaining penile erection, the search continues for more effective and more comfortable solutions. Nevertheless, there still remains a need for a device which can assist in and maintain the erection of a penis and which generally obviates the foregoing problems.

### OBJECTS OF THE INVENTION

Accordingly, the main object of the present invention is to provide a penile erection assisting device which generally assists in and maintains the erection of a penis.

Another object of the present invention is to provide a penile erection assisting device which generally reduces the risks of injuries to the genital organs.

Still another object of the present invention is to provide a penile erection assisting device which is generally easy to install and use and which is generally hygienic and easy to wash and clean before and after use.

Other and further objects and advantages of the present invention will be obvious upon an understanding of the illustrative embodiments about to be described or will be indicated in the appended claims, and various advantages not referred to herein will occur to one skilled in the art upon employment of the invention in practice.

### SUMMARY OF THE INVENTION

The present invention therefore provides a device for assisting in and maintaining penile erection, preferably during sexual intercourse. The device of the present invention generally comprises a flexible portion, preferably provided in the form of a band or a flat tubular section and a preferably elastic cord or string which is mounted to and which extends from each end of the flexible portion in order to define a preferably large loop.

The circumference of the loop so defined is preferably larger than the circumference of the waist of the user. Accordingly, during installation, the device can be positioned around the waist of the user, with the flexible portion at the user's back, in order to allow the flexible portion to be pulled between the legs of the user and then to be placed so as to engage the top of the penis, preferably at the base thereof.

Understandably, the length of the elastic cord is preferably adjustable in length with means known in the art in order to accommodate users of different sizes.

According to an important aspect of the present invention, the flexible portion is preferably made of natural and/or synthetic elastomeric material such as rubber or latex. In the preferred embodiment, the flexible portion is preferably made of latex.

By being made of such elastomeric and flexible material, the flexible portion is generally less likely to be displaced during sexual intercourse since it generally partially circumscribes and firmly engages the top portion of the base of the penis. Additionally, since the device is less prone to accidental displacements, the probability of injuries to the penis and/or to the testicles is greatly reduced.

Moreover, by being made of flexible material instead of rigid material as in the prior art, the flexible portion of the device of the present invention can generally automatically adjust itself to penis of different sizes, thereby providing a better adjustment.

In the end, the resulting device is one which is extremely effective for assisting in and maintaining erection of the penis without the problems associated with other prior art devices. The device is generally easy to use so as not to detract from sexual urge. It is especially helpful to those who have difficulty in maintaining an erection. However, for those who have no difficulty in maintaining an erection, it may simply be used to provide a more satisfying sexual encounter.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is a plan view of an embodiment of the device of the present invention.

FIG. 1A is a fractional view of a portion of the device of FIG. 1.

FIG. 2 is a fractional perspective view of the waist and genitalia of a human male with the device of FIG. 1 installed thereon.

FIG. 3 is a fractional side view of the waist and genitalia of a human male with the device of FIG. 1 installed thereon.

FIG. 4 is a fractional rear view of the waist and genitalia of a human male with the device of FIG. 1 installed thereon.

FIG. 5 is a fractional front view of the waist and genitalia of a human male with the device of FIG. 1 partially installed thereon.

FIG. 6 is a fractional front view of the waist and genitalia of a human male with the device of FIG. 1 fully installed thereon.

### DETAILED DESCRIPTION OF THE PREFERRED EMBODIMENT

A novel device for assisting in and maintaining penile erection will be described hereinafter. Although the invention is described in terms of specific illustrative embodiments, it is to be understood that the embodiments described herein are by way of example only and that the scope of the invention is not intended to be limited thereby.

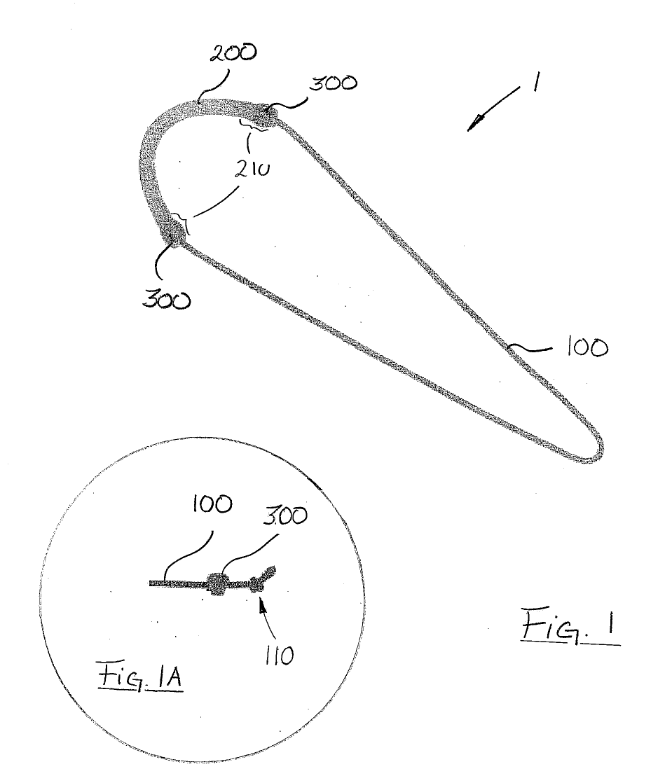

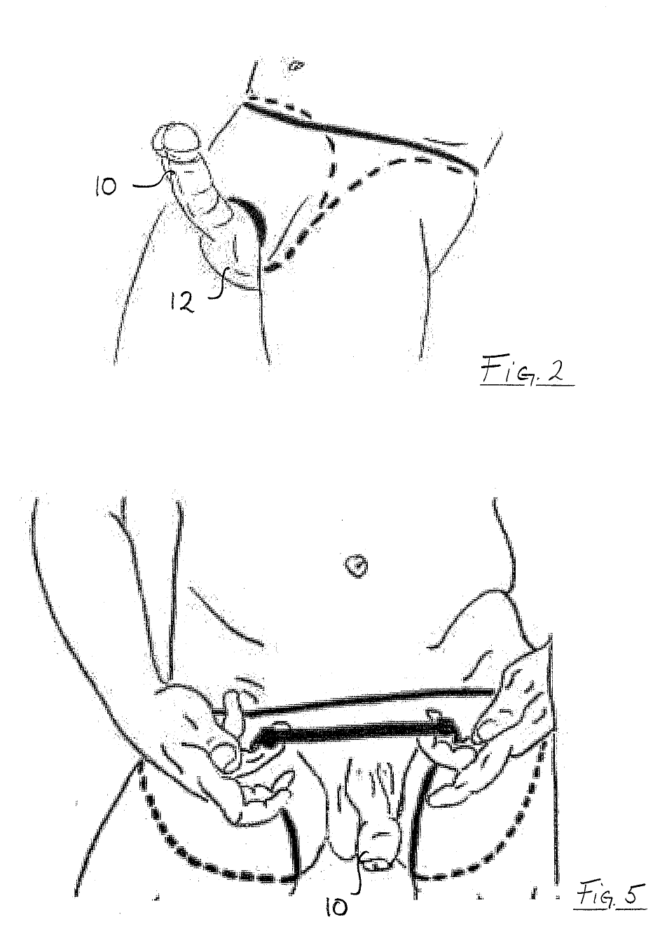

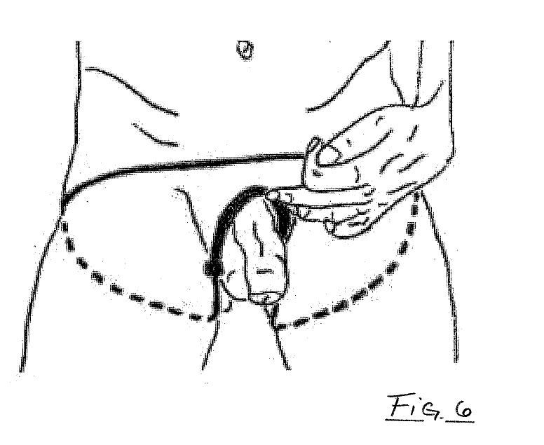

Referring first to FIG. 1, there is shown the device 1 of the present invention for assisting in and maintaining erection of the human penis 10 (shown in FIGS. 2 to 6). The device 1 comprises a relatively flexible portion 200 and an elastic cord or string 100 generally fixedly mounted to and extending from each end 210 of the flexible portion 200. When mounted together, the flexible portion 200 and the elastic cord 100 define a loop.

Understandably, the circumference of the loop so defined is preferably larger than the circumference of the waist of the user so that the device 1 can be adequately installed on the penis 10 it will be explained below.

The flexible portion 200 of the device 1 is generally made of natural and/or synthetic elastomeric material such as rubber or latex. In the preferred embodiment, the flexible portion is a section of latex tubing. Understandably, other material having material properties similar latex could be used without departing from the scope of the invention. Hence, the present invention is not limited to latex tubing.

Still referring to FIG. 1, the cord 100 is generally mounted to the portion 200 by frictional engagement. More particularly, as can be seen from FIG. 1A, each end portion of the cord 100 is equipped with protuberance means such as bead 300. The beads 300 have an aperture therethrough and are preferably maintained on the cord 100 by knots 110 which are larger than the diameter of the aperture of the beads 300. The bead equipped end portions of the cord 100 are then forcefully inserted into the tubular flexible portion 200, at the extremities 210 thereof. The friction between the latex tubing 200 and the beads 300 generally prevent the beads 300 from being expelled from the tubing 200.

Understandably, the skilled addressee would readily understand that other means to fasten the flexible portion 200 and the elastic cord 100 together are possible and that the invention is not limited to the exemplary embodiment described above.

Referring now also to FIGS. 2 to 6, the installation of the device 1 of the present invention will be described. With the device 1 formed in the loop fashion of FIG. 1 so as to provide a loop of a few centimeters greater than the circumference of the waist of the user, the cord 100 is placed around the user's waist first with the flexible portion 200 at the user's back. Then, the user reaches with one hand, from the front, through his crotch, grasping the flexible portion 200 and pulling it between its legs, placing the flexible portion 200 in engagement with the top of the penis 10 at the base thereof. In this position, as shown in FIGS. 2 to 6, the elastic cord 100 extends from the flexible portion 200 on each side of the user's scrotum 12, back between the legs and then up around the front of the waist.

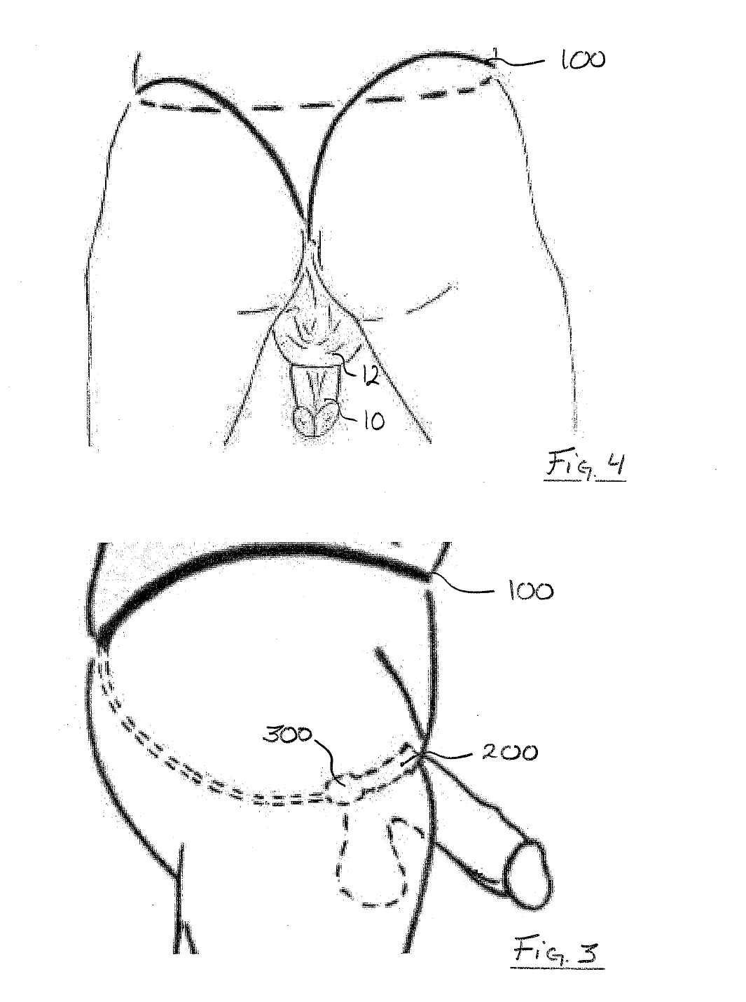

In the proper position of FIGS. 3 and 6, the flexible portion 200 therefore applies pressure to the upper portion of the penis 10 near the base thereof, restricting blood flow therefrom. This thus assists in and maintains the erection of the penis 10. However, since the flexible portion 200 does not totally encircle or circumscribe the penis 10, blood flow is not totally interrupted and neither is ejaculation prevented or hindered. The blood restriction results in the penis 10 being maintained at maximum rigidity until ejaculation.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A device to assist in and maintain the erection of a human penis, said device comprising:
a. a first elongated element, said first elongated element having first and second end portions and being made of flexible material;
b. a second elongated element, said second elongated element having third and fourth end portions and being made of elastic material;
wherein said third end portion is secure to said first end portion and said fourth end portion is secured to said second end portion, thereby defining a loop.

2. A device as claimed in claim 1, wherein said second elongated element is an elastic cord.

3. A device as claimed in claim 2, wherein the length of said elastic cord is adjustable.

4. A device as claimed in claim 1, wherein said flexible material is natural and/or synthetic elastomeric material.

5. A device as claimed in claim 1, wherein said flexible material is latex.

6. A device as claimed in claim 1, wherein said first elongated element is a flexible section of elastomeric tubing.

7. A device as claimed in claim 6, wherein said third and fourth end portions of said second elongated element are respectfully further equipped with first and second protuberance means.

8. A device as claimed in claim 7, wherein said first protuberance means is inserted into said first end portion of said flexible section of tubing, wherein said second protuberance means is inserted in said second portion of said flexible section of tubing and whereby said first and second protuberances means are frictionally engaged therein.

9. A device as claimed in claim 1, wherein said loop is adapted to be positioned around the waist of a user wherein said first elongated element is adapted to be positioned at the back of said user for allowing said first elongated element to be pulled between the legs of said user for placement in engagement with the top portion of said penis and at the base thereof.

10. A device to assist in and maintain the erection of the penis of a user, said device comprising:
a. a flexible first elongated element for engagement with the top portion of said penis at the base thereof, and
b. an elastic second elongated element for engagement at least with the waist of said user;
wherein said elastic second elongated element is mounted to and extends from each end of said flexible first elongated element in order to form a loop whereby said loop is adapted to be positioned around the waist of said user with said flexible first elongated element being positioned at the back of said user for allowing said flexible first elongated element to be pulled between the legs of said user for placement in engagement with said top portion of said penis at the base thereof.

11. A device as claimed in claim 10, wherein said elastic second elongated element is an elastic cord.

12. A device as claimed in claim 11, wherein the length of said elastic cord is adjustable.

13. A device as claimed in claim 10, wherein said flexible first elongated element is made of natural and/or synthetic elastomeric material.

14. A device as claimed in claim 10, wherein said flexible first elongated element is made of latex.

15. A device as claimed in claim 10, wherein said flexible first elongated element is a flexible section of elastomeric tubing.

16. A device as claimed in claim 15, wherein said flexible first elongated element comprises a first and a second end portions and wherein said elastic second elongated element comprises a third and a fourth end portions.

17. A device as claimed in claim 16, wherein said third and fourth end portions of said second elongated element are respectfully further equipped with first and second protuberance means.

18. A device as claimed in claim 17, wherein said first protuberance means is inserted into said first end portion of said flexible section of tubing, wherein said second protuberance means is inserted in said second portion of said flexible section of tubing and whereby said first and second protuberances means are frictionally engaged therein.
